(19) United States
(12) Patent Application Publication
(10) Pub. No.: US 2004/0010286 A1
(43) Pub. Date: Jan. 15, 2004

(54) Arthroscopic suture thread and method of use

(76) Inventor: Robert E. Gieringer, Anchorage, AK (US)

Correspondence Address: Robert M. Asher, Bromberg & Sunstein LLP, 125 Summer Street, Boston, MA, 02110-1618, US

(21) Appl. No.: 10/608,652

(22) Filed: Jun. 27, 2003

Related U.S. Application Data
(63) Continuation of application No. 09/811,144, filed on Mar. 16, 2001, now abandoned.
(60) Provisional application No. 60/189,800, filed on Mar. 16, 2000.

(51) Int. Cl. A61B 17/04

(52) U.S. Cl. 606/228

(57) ABSTRACT

An arthroscopic suture thread and method for using same with a suture punch system. The system having a passageway, a hollow needle at a distal end of the passageway, and a feeder wheel proximate to a proximal end of the passageway. The suture thread of one embodiment made from a non-bioabsorbable material includes a coupled, sufficiently stiff and long leader portion for permitting the feeder wheel to facilitate advancement along the passageway and through the hollow needle. The suture thread of a second embodiment is a braided non-bioabsorbable thread coated with a sufficiently stiff bioabsorbable material.

### RELATED U.S. APPLICATION

The present application is a continuation of U.S. patent application Ser. No. 09/811,144 filed Mar. 16, 2001 which claims priority from U.S. Provisional Application No. 60/189,800, filed Mar. 16, 2000, both of which are hereby incorporated herein by reference.

### TECHNICAL FIELD

This invention relates to surgical suture threads for arthroscopic use, and in particular, for use of such threads in conjunction with a surgical suture punch system.

### BACKGROUND ART

Suture threads have customarily been used to surgically repair separated tissues including simple superficial incisions and internal tears. With the recent introduction of arthroscopic techniques, the complexity typically associated with repairing separated internal tissues (such as a torn muscle or a torn cartilage) has been significantly reduced. For example, the size of the necessary incision has been substantially reduced without compromising the integrity of the repair made by the suture thread. Despite the advances however, a simple arthroscopic stitch has not always been easy to make to secure a tear site. Indeed, the inability to pass a suture thread across the separated tissue often frustrates even the most patient physician.

At present, there are available arthroscopic suturing devices which allow physicians to place a suture thread across a tear site of a separated tissue. One example of such a suturing device is the Shutt® suture punch system, as disclosed in Caspari, R. B., Arthroscopic reconstruction for anterior shoulder instability, Techniques Orthop., 3:(1):59-66 (1988). This system is also disclosed in U.S. Pat. No. 4,957,498, issued Sep. 18, 1990, to Caspari et al. Referring to FIG. 1, this particular suture punch system 10 (referred to hereinafter as “suture punch” or “arthroscopic suture punch”) is designed to offer physicians a method to arthroscopically place a suture thread 18 into targeted tissue. Suture punch 10 includes a passageway 12, a hollow needle 14 at a distal end 15 of passageway 12, and a feeder wheel 16 proximate a proximal end 17 of passageway 12. Such devices as arthroscopic suture punch 10 are positioned during arthroscopic procedures having proximal end 17 and, therefore, feeder wheel 16 located essentially external to an arthroscopic portal through which the remainder of the suture punch 10 is inserted. Generally, as suture thread 18 must be placed across the targeted tissue, hollow needle 14 is adapted to puncture and extend across the targeted tissue that is being accessed via the portal. The feeder wheel 16 is a rotatable mechanism for advancing the suture thread 18. It will be understood that needle 14 may, in general, be replaced with any hollowed shape capable of cutting or puncturing tissue. It will also be understood that known feeder mechanisms may be utilized to substitute for feeder wheel 16 of the Caspari design. The advancement of the suture thread 18 may be achieved by positioning the feeder wheel 16 so that it contacts, to a certain extent, a portion of the suture thread 18 at or near proximal end 17 of the passageway 12. The passageway 12, situated between the hollow needle 14 and the feeder wheel 16, acts as a guide for the advancing suture thread 18 to move from the feeder wheel 16 toward the distal end 15 and into and, subsequently, through hollow needle 14 and across the targeted tissue. Typically, a monofilament suture thread 18 is used in conjunction with suture punch system 10. Such a monofilament suture thread has sufficient stiffness to be advanced by the feeder wheel 16 along the passageway 12.

In addition, because the arthroscopic suture thread 18 must remain within a patient's body for an extended period to allow the targeted tissue to heal, the suture thread 18 for use with the arthroscopic suture punch system 10 is often made from a biocompatible and/or a bioabsorbable material. Bioabsorbable monofilament suture thread is unpredictable in its absorption rate. This is problematic because the suture may be absorbed prior to the complete healing of tissue. Alternatively, if a stiff, non-bioabsorbable monofilament suture thread (for instance, one made from nylon or polypropylene) is employed to avoid these problems, the tissue around which this thread is securely tightened may become irritated or otherwise damaged. Generally, stiff monofilament suture threads tend to cut or irritate delicate tissue.

### SUMMARY OF THE INVENTION

In accordance with an embodiment of the present invention, an arthroscopic suture thread capable of being fed through a suture punch is provided. The suture punch is designed to advance a sufficiently stiff suture through an arthroscopic portal. The suture punch has a passageway having a proximal end that is located essentially external to the portal during an arthroscopic procedure, a distal end that is located within the portal proximal to a targeted tissue during the procedure, a passageway length, and a hollow needle capable of cutting tissue through which the suture may pass. The needle is located essentially at the distal end. The arthroscopic suture thread has a flexible, braided portion capable of securing the targeted tissue. The thread also has a monofilament leader portion having a leader length. The monofilament leader portion is coupled to the flexible, braided portion, the leader length being longer than the passageway length, so that, during the procedure, the monofilament leader portion is advanced through the portal prior to advancement of the flexible, braided portion. The arthroscopic suture thread may have the leader length being at least as long as the braided length. The braided portion may be made from a non-bioabsorbable material which may be selected from Dacron®, nylon, and polypropylene. The leader portion may be made from a biocompatible material. The leader portion may be, alternatively, be made from a non-bioabsorbable material. The leader portion may be made from a material having stiffness at least equal to that of polypropylene. In an embodiment, the braided portion and the leader portion have approximately the same diameter. The thread may, in another embodiment, be manufactured by connecting the braided portion to the monofilament leader portion by heat welding.

In another embodiment, an arthroscopic suture thread capable of being fed through a suture punch is provided. The suture punch has a passageway, a hollow needle at a distal end of the passageway, and a feeder wheel proximate a proximal end to the passageway. The arthroscopic suture thread has a braided suture coated with a bioabsorbable composition for providing the arthroscopic suture thread with a sufficient stiffness so as to allow the feeder wheel to facilitate, by rotation, advancement of the suture thread along the passageway and through the hollow needle. The braided suture may be made from a non-bioabsorbable material which may be Dacron®. The bioabsorbable composition may be polydioxanone.

In yet another embodiment, an arthroscopic suturing system is provided. The system has a suture punch having a passageway, a hollow needle at a distal end of the passageway, and a feeder wheel proximate a proximal end of the passageway, the feeder wheel capable of advancing a sufficiently stiff suture thread along the passageway and through the hollow needle. The system also has a suture thread comprising a multi-filament, flexible, non-bioabsorbable portion and a leader portion, the leader portion stiffer than the flexible portion, the leader portion longer than the passageway, so that the feeder wheel is capable of, by rotation, advancing the suture thread along the passageway and out through the hollow needle. The multi-filament, flexible portion may be made from Dacron®. The multi-filament, flexible portion and the leader portion of the suture thread may be similar in diameter. The flexible portion of the suture thread may be coupled to the leader portion by heat welding. The leader portion may be made from a material having stiffness at least equal to that of polypropylene.

Further embodiments include methods of arthroscopically suturing tissues.

### BRIEF DESCRIPTION OF THE DRAWINGS

FIG. 1 shows a prior art suture punch for use with an arthroscopic suture thread.

FIG. 2 illustrates an arthroscopic suture thread having a braided portion and a leader portion in accordance with an embodiment of the invention.

FIG. 3 is a partial view of the suture thread of FIG. 2 illustrating a connection between the leader portion and the braided portion, in accordance with an embodiment.

FIG. 4 illustrates a braided arthroscopic suture thread having a coating in accordance with another embodiment.

FIG. 5 is a partial view of the suture thread of FIG. 4 detailing the coating on the suture.

FIG. 6 is an isometric end view of the proximal end of the prior art suture punch of FIG. 1, illustrating an embodiment of the arthroscopic suture thread inserted into the suture punch.

FIGS. 7 and 8 illustrate, in series, steps for arthroscopically suturing tissues using a suture thread, in accordance with an embodiment, in conjunction with the suture punch of FIG. 1.

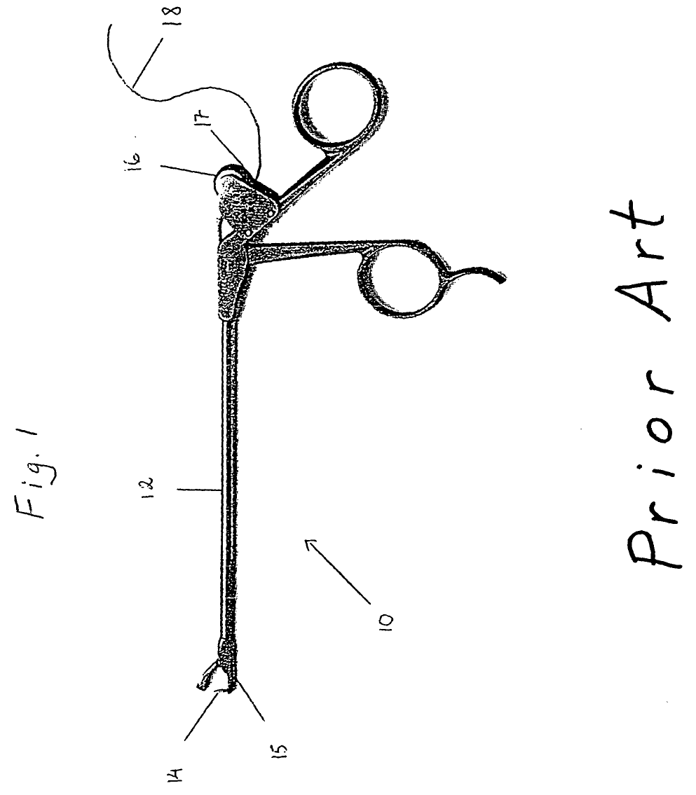

### DETAILED DESCRIPTION OF SPECIFIC EMBODIMENTS

It is desirable to utilize an arthroscopic suture thread for use with a arthroscopic suture punch that not only is sufficiently stiff so as to allow it to be advanced along the passageway of the suture punch, but one that also has sufficient strength and durability to allow the suture thread to be securely pulled and tightened around a tear site without damaging the targeted tissue, while allowing the tissue to properly heal.

FIG. 2 illustrates an embodiment of an arthroscopic suture thread 20 that may be used in conjunction with the above-described suture punch 10 to repair separated tissues. Such tissues should, naturally, be susceptible to penetration by the hollow needle 14 of suture punch 10. In accordance with one embodiment of the present invention, suture thread 20 comprises a soft braided portion 22 and a stiff leader portion 24 coupled to one end of the braided portion 22.

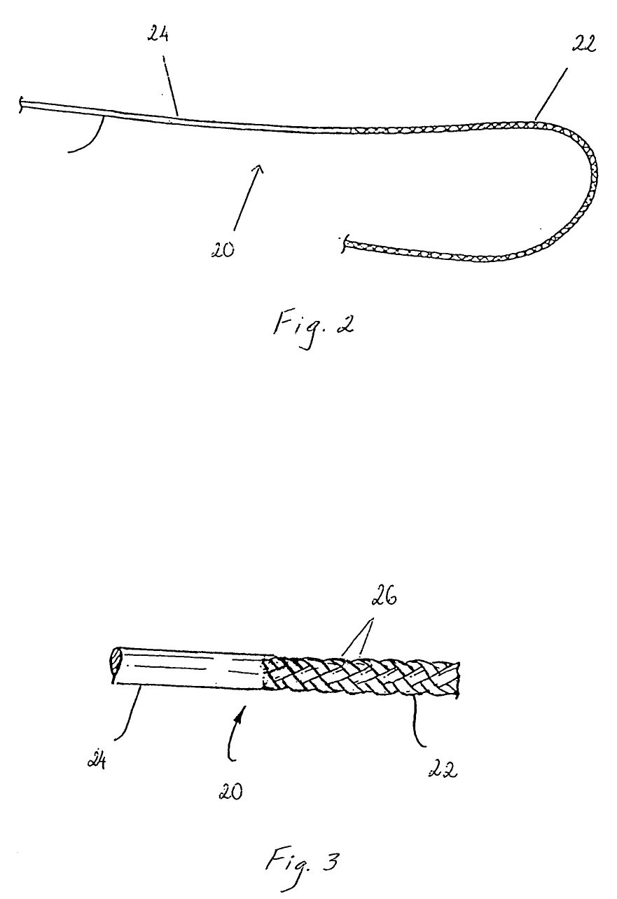

FIG. 3 is a partial view of suture thread 20 showing the connection between the braided portion 22 and the leader portion 24. The braided portion 22 is designed primarily for tissue securing purposes. To this end, the braided portion 22 is preferably biocompatible, easily deformable, and strong. To ensure that the braided portion 22 is easily deformable, in one embodiment of the present invention, braided portion 22 is made with a substantially flexible material along its entire length. The flexibility or pliability of braided portion 22 is enhanced by providing many filaments 26 of material and twisting or braiding them together as shown in FIG. 3. The small diameter of filaments 26 tends to make them highly pliable. The flexibility of the braided portion 22 is advantageous as it permits the braided portion 22 to loop around the targeted tissue (for example, a torn rotator cuff) to secure fragments of the targeted tissue against one another. Furthermore, as the forces exerted on braided portion 22 may be substantial during tightening, as can be seen from FIG. 3 braided portion 22 is provided with a plurality of filaments 26 so that the physical integrity of the suture thread 20 may be maintained. In accordance with an embodiment of the invention, braided portion 22 has a strength of approximately 20 pounds. The braided portion 22 may be further designed to secure targeted tissue fragments for a longer time period than may be necessary for the tissue to heal. Braided portion 22 may be made from a biocompatible, yet non-bioabsorbable material such as Dacron®, nylon, or polypropylene.

The flexibility of the braided portion is indeed desirable. However, this flexibility can and usually will interfere with advancement of the suture thread 20 along the passageway 12 of suture punch 10. In particular, the flexible braided portion 22 may instead bunch up within the passageway 12. An arthroscopic suture thread 20 embodiment may include a leader portion 24 having sufficient stiffness and strength at one end of the braided portion 22 (see FIG. 3). By having a sufficient stiffness, the leader portion 24 may maintain an elongated shape to permit its advancement by the feeder wheel 16 along the passageway 12 so as to pull the braided portion 22 therewith. The stiffness of leader portion 24 may desirably be at least equal to the stiffness of polypropylene fiber.

The strength of leader portion 24 prevents any compromise in the integrity of the leader portion 24 while for example, the leader portion 24 is pulled, by the suture punch 10, away from the targeted tissue. The leader portion 24 may be provided with a strength falling within a range from about 10 pounds to about 20 pounds. Although particularly stiff and strong, the leader portion 24 is, nevertheless, desirably bendable. Its ability to bend further permits the leader portion 24 to be manipulated with suture punch 10. More specifically, bendability aids in the advancement of thread 20 to transition from passageway 12 into the hollow needle 14 without the need for additional guiding or other mechanisms. The needle 14 is situated relatively perpendicular to passageway 12.

In order to exit from hollow needle 14 and move across the targeted tissue, leader portion 24, in accordance with one embodiment of the invention, must be relatively longer than passageway 12. Otherwise, before leader portion 24 may exit hollow needle 14, the feeder wheel 16 may engage the flexible braided portion 22. If this were to occur, it would make advancement of the suture thread 20 almost impossible. Moreover, for ease of use with the suture punch 10, it is preferable that the leader portion 24 and the braided portion 22 be at least comparable in length. Thus, once leader portion 24 has been advanced across the targeted tissue, the braided portion 22 may be adequately available for tissue securing purposes. Leader portion 24, unlike braided portion 22, is not adapted for securing purposes. As such, it may have only a single filament rather than multiple filaments. Nonetheless, as the leader portion 24 must move through the targeted tissue, it is preferable that the leader portion 24 be biocompatible. In one embodiment, a monofilament leader portion 24 is made from a non-bioabsorbable, yet biocompatible material. The non-bioabsorbable material may be selected from suitable plastics such as nylon or polypropylene. As an example, a Dacron® leader portion 24, may be used when made as a monofilament having a thickness corresponding approximately to that of the braided portion 22.

As both the leader portion 24 and the braided portion 22 must move across the targeted tissue, in order to provide suture thread 20 with a continuous, gliding movement, portions 22 and 24 may each be formed with a substantially similar diameter. By providing the two portions 22 and 24 with a similarly sized diameter, it is possible to prevent the suture thread 20 from getting caught within a tear site and, thereby, avoiding unnecessary damage to the targeted tissue. Moreover, as the leader portion 24 and the braided portion 22 must both exit through the hollow needle 14, the leader portion 24 and the braided portion 22 should be made with a diameter smaller than the opening in the needle 14 through which they must advance.

Referring again to FIG. 3, the leader portion 24 and the braided portion 22 are arranged in an end to end configuration. In this configuration, portions 22 and 24 may be attached to one another via any biocompatible manner known in the art. In an embodiment, an end of the leader portion 24 may be heat welded to an end of the braided portion 22 in such a way that there is a continuous transition between the two portions. Using this forming method, the point of connection between the leader portion 24 and the braided portion 22, maintains a diameter similar to the diameter of each of the two portions 22 and 24.

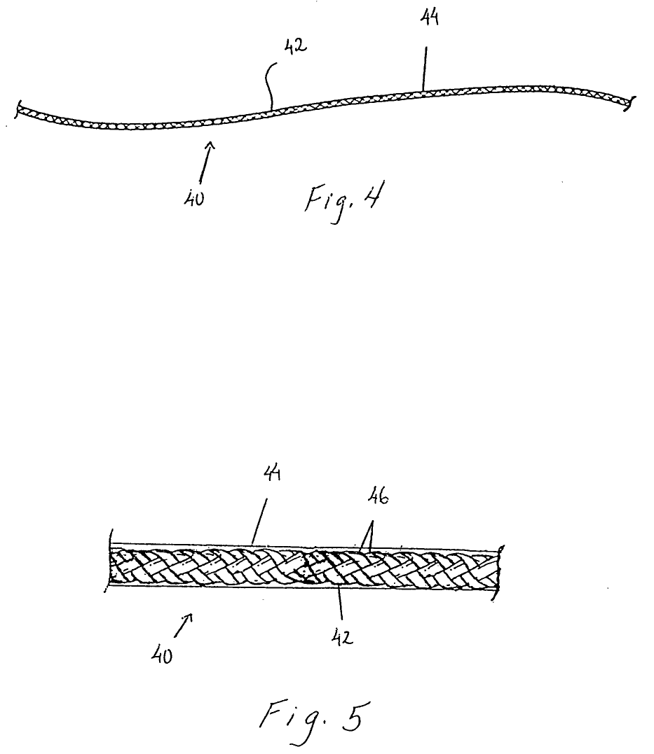

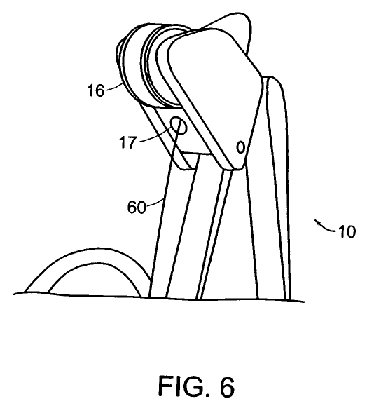

FIG. 4 illustrates a suture thread 40 in accordance with another embodiment of the present invention. Suture thread 40, also adapted for use with above-described suture punch 10, comprises a braided suture 42 having a coating 44 (see FIG. 5) along its entire length. Braided suture 42, similarly to the aforementioned braided portion 22 of suture thread 20, is designed primarily for tissue securing purposes. To this end, braided suture 42 may be biocompatible yet deformable so as to be able to loop itself around a targeted tissue (for instance, a torn rotator cuff) to tightly secure the tissue. Furthermore, as the braided suture 42 must withstand significant forces sometimes exerted during tightening, braided suture 42, may have a plurality of filaments 46 to reduce the chance of breakage. Filaments 46, manufacturable from a durable material that is substantially flexible, may be twisted together or braided to provide braided suture 42 with a strength measuring approximately 20 pounds. In addition (and similar to filaments 26 in the aforementioned braided portion 22), filaments 46 are substantially similar in diameter and, preferably, total at least three in number. Filaments 46 may be made from biocompatible, non-bioabsorbable Dacron®.

Still referring to the embodiment of FIG. 5, the braided suture 42 is shown having a coating 44 along its entire length. The particular application of coating 44 is necessitated by the overall lack of stiffness of braided suture 42 which tends to make the advancement of the suture thread 40 within the passageway 12 difficult. In utilizing coating 44 of this embodiment, the braided suture 42 is provided with a sufficient stiffness necessary to allow the suture thread 40 to advance within the passageway 12. However, it should be noted that while the coating only causes the braided suture 42 to be less flexible in a linear direction, the braided suture 42 retains an ability to bend from side to side. In this manner, interference with the capability of the suture thread to tightly secure a targeted tissue is minimized.

Coating 44 may be applied onto braided suture 42 by methods known in the art. One example is via dip coating. Here, suture thread 40 is dipped into a solution of the coating composition before being removed to dry. Coating 44 may, for example, be made from a biocompatible and bioabsorbable composition such as polydioxanone. Coating 44, in addition, must not be so thick in diameter that it prevents suture thread 40 from moving through hollow needle 14 and across the targeted tissue.

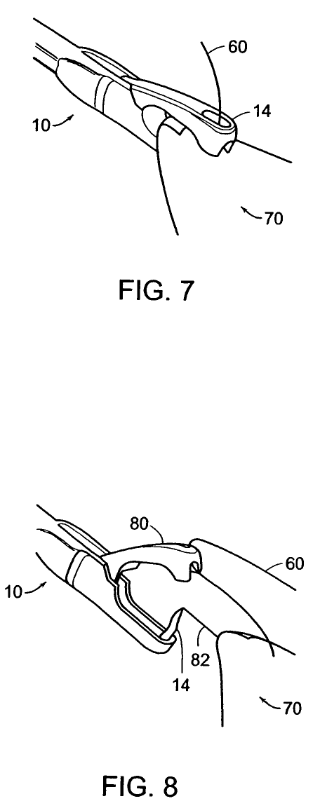

FIGS. 6-8 illustrate the use of either embodiment of suture thread (20 or 40) denoted 60 with suture punch 10. Initially, as shown in FIG. 6, suture thread 60 must be inserted into the suture punch 10 through the proximal end 17 of the passageway 12. Thereafter, the feeder wheel 16 may be rotated in a direction away from the distal end 15 to partially advance the suture thread 60 down the passageway 12. In FIG. 7, a targeted tissue 70 is shown engaged by the suture punch 10 and punctured by the needle 14. Once the tissue 70 has been punctured, the needle 14 is allowed to remain extended there across, and the feeder wheel 16 may again be rotated in a direction away from the distal end 15 to urge the suture thread 60 through the needle 14 and across the targeted tissue 70. The rotation of the feeder wheel 16 may continue until an adequate portion of the suture thread 60 has been advanced across the tissue 70. Subsequently, referring now to FIG. 8, the needle 14 may be disengaged and the suture punch 10 pulled away from the tissue 70. It can be appreciated that one end of the suture thread 60 is lodged against an upper jaw portion 80 of suture punch 10 when the suture punch 10 is pulled away from the tissue 70, while the remainder of the suture thread 60 is caused to slide through the needle 14 to form a loop 82 across the tissue 70. Thus, the further suture punch 10 moves away from tissue 70, the more of the suture thread 60 is caused to move out the passageway 12, through the needle 14 and across the tissue 70. Once the suture punch has been withdrawn, for example, from within a patient, the suture thread 60 may be removed from the suture punch 10 and tightly secured against the tissue 70. If, for instance, a suture thread with an embodiment similar to suture thread 20 is used, the leader portion 24 should be cut off separating it from the braided portion 22 prior to the securing of the suture thread 20 against the tissue 70.

Although the invention has been described with reference to several preferred embodiments, it will be understood by one of ordinary skill in the art that various modifications can be made without departing from the spirit and the scope of the invention, as set forth in the claims hereinbelow.

What is claimed is:

1. An arthroscopic suture thread comprising:
a flexible, braided portion capable of securing the targeted tissue; and
a monofilament leader portion having a leader length, the monofilament leader portion coupled to the flexible, braided portion, the leader length being longer than an elongated passageway of a suture punch, so that when used with the suture punch, the monofilament leader portion is fed through the elongated passageway ahead of the flexible, braided portion.

2. An arthroscopic suture thread according to claim 1, wherein the flexible, braided portion has a braided length, the leader length being at least as long as the braided length.

3. An arthroscopic suture thread according to claim 1, wherein the braided portion is made from a non-bioabsorbable material.

4. An arthroscopic suture thread according to claim 3, wherein the non-bioabsorbable material is selected from Dacron®, nylon, and polypropylene.

5. An arthroscopic suture thread according to claim 4, wherein the non-bioabsorbable material is Dacron®.

6. An arthroscopic suture thread according to claim 3, wherein the leader portion is made from a biocompatible material.

7. An arthroscopic suture thread according to claim 3, wherein the leader portion is made from a non-bioabsorbable material.

8. An arthroscopic suture thread according to claim 3, wherein the leader portion is made from a material having stiffness at least equal to that of polypropylene.

9. An arthroscopic suture thread according to claim 1, wherein the braided portion and the leader portion have approximately the same diameter.

10. An arthroscopic suture thread according to claim 1, wherein the thread is manufactured by connecting the braided portion to the monofilament leader portion by heat welding.

11. An arthroscopic suture thread comprising a braided suture coated with a bioabsorbable composition for providing the arthroscopic suture thread with a sufficient stiffness to allow the suture thread to be pushed through a passageway and hollow needle of a suture punch.

12. An arthroscopic suture thread according to claim 11, wherein the braided suture is made from a non-bioabsorbable material.

13. An arthroscopic suture thread according to claim 12, wherein the non-bioabsorbable material is Dacron®.

14. An arthroscopic suture thread according to claim 11, wherein the bioabsorbable composition is polydioxanone.

15. An arthroscopic suturing system comprising:
a suture punch having a passageway and a hollow needle at a distal end of the passageway; and
a suture thread comprising a multi-filament, flexible, non-bioabsorbable portion and a leader portion, the leader portion being stiffer than the flexible portion and longer than the passageway to permit pushing the suture thread along the passageway and out through the hollow needle.

16. An arthroscopic suturing system according to claim 15, wherein the multi-filament, flexible portion is made from Dacron®.

17. An arthroscopic suturing system according to claim 15, wherein the multi-filament, flexible portion and the leader portion of the suture thread are similar in diameter.

18. An arthroscopic suturing system according to claim 15, wherein the flexible portion of the suture thread is coupled to the leader portion by heat welding.

19. An arthroscopic suturing system according to claim 15, wherein the leader portion is made from a material having stiffness at least equal to that of polypropylene.

20. A method for arthroscopically suturing tissues comprising:
placing a suture punch through an arthroscopic portal, the punch having a passageway, a hollow needle at a distal end of the passageway, the distal end placed proximal to tissues to be secured, and a feeder wheel proximate a proximal end to the passageway, the suture punch designed to advance a sufficiently stiff suture through the portal;
providing an arthroscopic suture thread comprising a flexible, multi-filament non-bioabsorbable portion for fixation and security of tissues, and a monofilament leader portion coupled to an end of the flexible, multifilament non-bioabsorbable portion, the leader portion being longer than the passageway and sufficiently stiff so as to allow the feeder wheel to facilitate, by rotation, advancement of the leader portion along the passageway and out through the hollow needle;
inserting the leader portion into the passageway of the suture punch;
puncturing the needle across the tissues;
rotating the feeder wheel to advance the leader portion along the passageway until a segment of the leader portion is pushed out through the needle and across the tissues; and
drawing the leader portion away from the tissues until a segment of the braided portion has passed through the tissues.

21. A method according to claim 20 further comprising:
removing the leader portion from the multi-filament, non-bioabsorbable portion.

22. A method according to claim 20 further comprising:
disengaging the needle from the tissues; and
pulling the suture punch away from the tissues.

23. A method for arthroscopically suturing tissue comprising:
providing a suture punch having a passageway and a hollow needle at a distal end of the passageway;
providing an arthroscopic suture thread including a braided non-bioabsorbable suture having a bioabsorbable composition coating for providing a sufficient stiffness so as to allow the suture thread to be pushed through the passageway and out through the hollow needle;
inserting a coated end of the suture thread into the passageway;
puncturing the needle across the tissues; and
advancing the suture thread along the passageway until a segment of the suture thread is pushed through the needle and across the tissues.

24. A method according to claim 23 further comprising:
disengaging the needle from the tissues; and
pulling the suture punch away from the tissues.

25. A method according to claim 23 wherein the suture punch further comprises a feeder wheel proximate a proximal end of the passageway and wherein advancing the suture thread comprises rotating the feeder wheel to advance the suture thread.

26. An arthroscopic suturing system according to claim 15 wherein the suture punch further comprises a feeder wheel proximate a proximal end of the passageway which upon rotation advances the suture thread along the passageway and out through the hollow needle.
